(19) United States
(12) Patent Application Publication
(10) Pub. No.: US 2007/0038304 A1
(43) Pub. Date: Feb. 15, 2007

(54) Flexible acetabular cup and method of manufacture thereof

(75) Inventors: Patrick Raugel, Ramsey, NJ (US); Eric Jones, Limerick (IE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 11/406,688

(22) Filed: Apr. 19, 2006

(30) Foreign Application Priority Data
Apr. 19, 2005 (GB) 0507884.5

(51) Int. Cl. A61F 2/34

(52) U.S. Cl. 623/22.3

(57) ABSTRACT

A flexible acetabular cup is provided which has an inner bearing surface which comprises a substantially part-spherical portion and two independent arms projecting therefrom. The arms form a separation or opening in the rim of the cup with the external surface of the cup being shaped to cause the independent arms to be pre-stressed towards each other when the cup is inserted in place in a pre-prepared acetabular socket. One way of accomplishing this is to form the cup with the arms bowed outwardly at the time of manufacture.

### BACKGROUND OF THE INVENTION

This invention relates to a flexible acetabular cup and a method of manufacture thereof.

Flexible acetabular cups are known, the inner bearing surfaces of which comprise a substantially part-spherical portion and two independent arms projecting therefrom and formed by a separation or opening in the rim of the cup.

Flexible acetabular cups of this type are shown in U.S. Pat. Nos. 5,609,646 and 6,758,864, the disclosures of which are incorporated herein by reference.

Cups of this type have a polar area in a part-spherical portion which carries the main load and the part-spherical portion can be relieved over part of its surface and the arms.

With flexible cups of this type the flexibility can cause the risk of deformation at the polar contact area so that the cup deforms and creates only ring contact which is undesirable.

Prior to fitting of the cup the acetabular socket is reamed and the tolerances can leave a difference of 0 mm to 0.6 mm between the socket and the cup so it may not be a perfect fit.

It has been found that the cup can be arranged to be inserted with an interference fit of up to about 0.6 mm but above that it as been found that the interference causes too much deformation and again creates a ring contact.

The present invention is intended to provide a construction and method of making a flexible acetabular cup which overcomes the difficulties concerned.

### SUMMARY OF THE INVENTION

According to the present invention a flexible acetabular cup, the inner bearing surface of which comprises a substantially part-spherical portion and two independent arms projecting therefrom and which are formed by a separation or opening in the rim of the cup, and in which there is a polar bearing area in the part-spherical portion and part of the part-spherical portion and the arms is relieved, characterized in that the external surface of the cup is shaped to cause the independent arms to be pre-stressed towards each other when the cup is inserted in place in a pre-prepared acetabular socket.

Thus, the external cordal-dimensions of the cup across the separation or opening between the arms can be greater than the external diameter of the part-spherical portion so that when the cup is placed in position in a pre-prepared part-spherical socket the arms are pre-stressed inwardly towards the separation between them.

The separation between the arms can be greater by 0.3 mm to 0.5 mm than the external diameter of the part-spherical portion.

It has been found that the pre-stressing of the arms reduces deformation and acts to prevent the polar area of contact degenerating into ring contact.

The inventions also includes a process for making the flexible acetabular cup as set forth above by including the steps of:
molding the cup from a synthetic plastics material to provide the shape with the two arms;
placing the molded cup, whilst still warm, in a jig which has a separator, the dimensions of which are greater than the separation or opening between the arms;
applying pressure to the cup to force the separator into the separation or opening to force the arms apart;
and, allowing the cup to cool.

Thus the separator can distort the arms by between 0.1 mm and 1 mm and preferably between 0.3 mm to 0.5 mm from the original molded shape so that the arms pre-stress when the cup is placed in the prepared acetabulum.

The invention can also include a method of fitting a flexible acetabular cup into an acetabular socket which includes machining the socket to provide a part-spherical recess and inserting a flexible cup as set forth above under pressure to cause the arms to deform towards each other so that they are pre-stressed toward each other when in place.

### BRIEF DESCRIPTION OF THE DRAWINGS

The invention can be performed in various ways and one embodiment will now be described by way of example and with reference to and as shown in the accompanying drawings.

FIGS. 1, 2, and 3 are respectively side, front, and top elevations of a flexible cup of the type described in U.S. Pat. No. 6,758,864;

FIG. 4 is a diagrammatic illustration showing how deformation of a flexible cup can cause ring contact on a ball head;

FIG. 5 is a plan view of the cup shown in FIGS. 1, 2, and 3 showing how it can be shaped during manufacture;

FIG. 6 is a diagrammatic side elevation of the cup shown in FIGS. 1, 2, 3, and 4 in place on a deformation jig.

The cup can be made from a single material, for example (CFR PEEK) as described in U.S. Pat. No. 6,758,864, or from two or more materials as set forth in U.S. Pat. No. 5,609,646.

### DETAILED DESCRIPTION

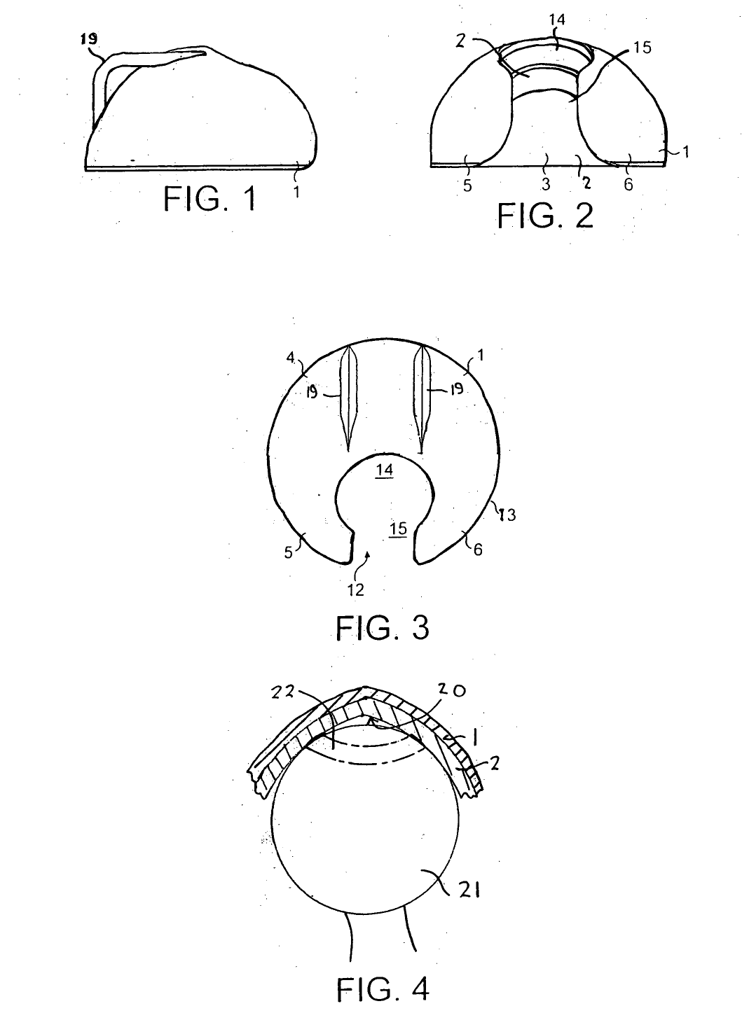

The cup shown in FIGS. 1, 2, and 3 is similar to that described in U.S. Pat. No. 6,758,864 and comprises a backing 1 which supports a bearing liner 2. The bearing liner has a bearing surface 3 and the backing is molded from a relatively rigid polymeric material, for example polyurethane in the form of Corothane 750D, CFR-PBT, CFR-PEEK or polymethylmethacrylate. The liner can be made from Corothane 80A and the liner and backing can be molded together by the technique described in U.S. Pat. No. 5,879,387, the liner being bonded to the backing by the molding process. The disclosure of U.S. Pat. No. 5,879,387 is incorporated herein by reference.

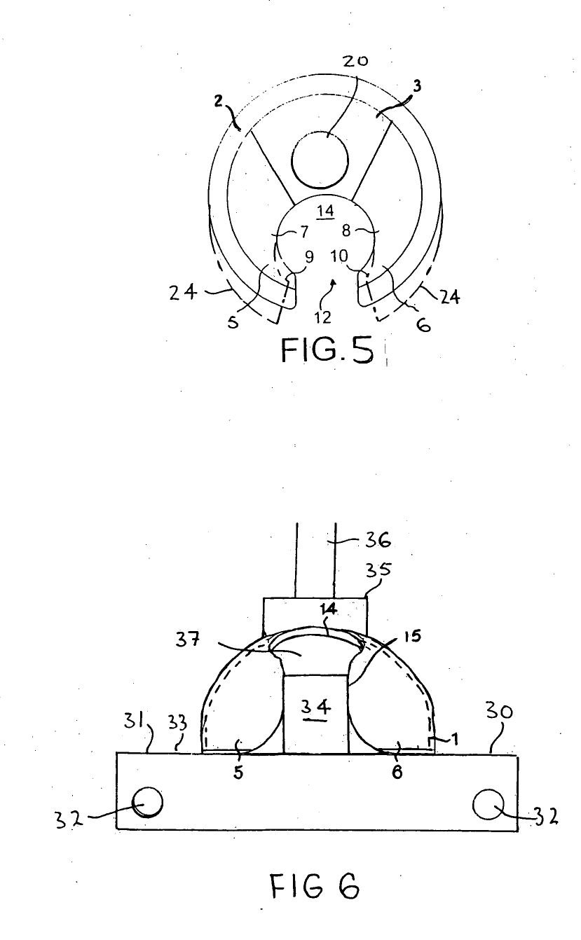

The overall shape and construction of the cup is similar to that described in U.S. Pat. No. 5,609,646, that is the main portion 4 of the backing is substantially part-spherical and there are two independent arms 5 and 6 which extend from the main part 4. The external shape of the inner bearing liner is also hemispherical and fits within the backing 1 but the inner surface of the bearing component is only hemispherical over its main portion and is relieved over the inner surfaces 7 and 8 of its arms 9 and 10, best shown in FIG. 5.

If desired the inner bearing liner can be substantially hemispherical and merely carry a thin splitting line to provide the two arms but in the construction shown in the drawings the arms 5 and 6 and 9 and 10 are spaced apart to provide a gap or opening 12 between them. The arms are spaced apart about an arc on the part-spherical main portion of the backing and the liner breaking out on the rim 13 and the outer surface of the arms themselves and the main portion are together substantially part-spherical.

Backing 1 thus comprises a substantially part-spherical wall having a rim 13 which is interrupted by a shaped opening to provide two spaced apart arms 5 and 6.

The main part 14 of the opening 12 is substantially semi-circular and has a mouth 15 which provides the interruption in the rim 13 and which is of smaller width than the remainder 14 of the opening. The backing 1 is therefore substantially horseshoe-shaped.

Because of its construction and the materials used the backing is sufficiently flexible to accept deformation of the acetabulum of the patient but is stiffer than the inner bearing component.

In the preferred construction shown in the drawings the outer surface of the backing 1 does not include the projecting strakes of U.S. Pat. No. 6,758,864 and the surface is substantially part-spherical apart from a pair of projecting fins 19 which extend in spaced apart parallel cordal directions.

The outer surface of the backing is preferably plasma sprayed with hydroxyapatite (HA) coating which is osteo-conductive and stimulates bone growth. The cup can also be made as set forth in UK Patent Application No. 04 22066.8 and have a metal backing layer, for example titanium, tantalum or niobium or even pure PEEK, to produce a barrier between composite material and the bone cells.

In order to insert the acetabular cup into an acetabulum, the bone is first prepared to the appropriate shape and a series of triangular grooves are carved into it by the surgeon which will line up with and accept fins 19.

The cup is now placed in position and tapped, the fins 19 holding it accurately as required. It has been found that the pressure of the ball of the femoral prosthesis or the natural ball shape of the bone acts to pressure the cup to hold it in position while bone growth takes place.

With this arrangement it is desirable to provide certain tolerances but it has been found that there is a risk of deformation at the polar contact area (indicated by reference numeral 20 in FIG. 5) so that the cup deforms and creates only ring contact which is undesirable and provides excessive wear and high frictional torque. This can take place with cups as described above or cups having plain outer surfaces or made from a single material. In FIG. 4 the deformation is exaggerated to explain the problem.

The effect of the deformation is shown diagrammatically in FIG. 4. The polar area 20 of the cup 1 deforms upwardly so that the contact with the ball, indicated by reference numeral 21, is reduced to a ring or line contact, indicated by chain lines 22. Such ring contact is undesirable and provokes wear on the surface as referred to above.

FIG. 5 is a plan view of the construction shown in FIGS. 1, 2 and 3 and shows how the arms 5 and 6 can be pre-shaped which causes them to be pre-stressed inwardly towards opening 12 when fitted into a prepared part-spherical acetabular socket.

The cup is molded initially to the shape shown in full lines in FIGS. 1, 2, 3 and 5. It is then deformed to the shape indicated by chain lines 24 in FIG. 5. In order to achieve the deformation a jig 30 is employed and which comprises a base 31 provided with cooling water channels 32. The upper surface 33 of the base is substantially flat or shaped to accept the outer periphery of the rim of the cup 1 with which it is to be used. A separator block 34 is provided on the base 30 and above it is located a pressure pad 35 which can be pressed downwardly by a pressure arm 36. The cup is located on a domed boss 37 also provided on the base 30.

After the cup has been initially molded, but whilst it is still warm, it is placed in the jig 30. The width of the block 34 is greater than the width of the opening 12 of the molded cup by between 0.1 mm and 1 mm and preferably between 0.3 mm and 0.5 mm. With the cup placed on the block 34 pressure is applied to the pressure pad 35 which forces the cup over the block thus distorting the arms 5 and 6 so that they are pressured outwardly to the position shown by broken lines 24 in FIG. 5. It will be appreciated that the distances shown in FIG. 5 are exaggerated to explain the process.

With the cup held in this position it is then allowed to cool assisted by the water cooling in the channels 32.

The removed cup is of the shape shown in chain lines in FIG. 5.

In order to install the cup in a prosthetic socket the socket is machined to a hemispherical shape it will be appreciated that the distortion has caused the arms to expand slightly in relation to the remaining portion of the cup so that it is no longer part-spherical but of a slightly deformed shape. When the cup is now placed in the part-spherical machined socket the arms are pressed towards each other so that the cup is pre-pressurized.

It has been found that with this pre-pressurization and with an interference fit effect of up to 0.6 mm deformation of the cup in the polar area is prevented as is the creation of ring contact.

It will be appreciated that in the description of the device and apparatus shown above a particular embodiment of cup is referred to but the invention can be applied to any cup of the type which has the two separated arms and which can have a variety of types of construction or shape.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A flexible acetabular cup comprising an inner bearing surface having a substantially part-spherical portion and two independent arms projecting therefrom, said arms spaced to form a separation or opening in the rim of the cup, the cup having an external surface in which an external cordal-dimensions of the cup across the separation or opening between the arms is greater than an external diameter of the part spherical portion to cause the independent arms to be pre-stressed towards each other when the cup is inserted in place in a pre-prepared acetabular socket.

2. The flexible acetabular cup as claimed in claim 1 in which the separation gap between the arms is greater by between 0.1 mm and 1 mm than the external diameter of the part-spherical portion.

3. The flexible acetabular cup as claimed in claim 2 in which the separation gap between the arms is greater by between 0.3 mm and 0.5 mm than the external diameter of the part-spherical portion.

4. The flexible acetabular cup as claimed in claim 1 in which the cups made at least in part from carbon fiber reinforced polyetheretherketane (PEEK).

5. The flexible acetabular cup as claimed in claim 4 in which the cup is made from two materials and has a bearing liner and a backing molded from a rigid polymeric material, for example polyurethane in the form of Corothane 80A.

6. The flexible acetabular cup as claimed in claim 5 in which the backing material is Corothane 750D, CFR-PBT, CFR-PEEK or polymethylmethacrylate.

7. The flexible acetabular cup as claimed in claim 4 in which the cup is made from two materials and has a metal or synthetic plastics material backing layer.

8. The flexible acetabular cup as claim in claim 7 in which the backing layer is titanium, tantalum or niobium.

9. The flexible acetabular cup as claimed in claim 7 in which the synthetic plastics material backing layer is made from pure PEEK.

10. The flexible acetabular cup as claimed in claim 5 in which the bearing liner is made from a composite material.

11. The flexible acetabular cup as claimed in claim 5 which has an outer surface which has been plasma sprayed with a hydroxyapatite (HA) coating.

12. A process for making the flexible acetabular cup as set forth in claim 1 comprising:
molding the cup from a synthetic plastics material to provide the shape with the two arms;
placing the molded cup, while still warm, in a jig which has a separator, the dimensions of which are greater than the separation or opening between the arms;
applying pressure to the cup to force the separator into the separation or opening to force the arms apart;
and, allowing the cup to cool.

13. The process as claimed in claim 12 in which the separator is used to force the arms apart between 0.1 mm and 1 mm from the original molded shape.

14. The process as claimed in claim 13 in which the arms are forced apart between 0.3 mm and 0.5 mm from the original molded shape.

15. A flexible acetabular cup comprising:
a part-spherical surface defined by a radius R extending from a center point, said part-spherical surface having an open portion thereon defining a pair of arms spaced along latitudes of said part-spherical surface, a portion of each of said arms along a latitude adjacent said open portion spaced from said center point a distance greater than R.

16. The flexible acetabular cup as set forth in claim 15 wherein the distance said portion of each of said arms is spaced from said center points between 0.1 and 1 mm greater than R.

17. The flexible acetabular cup as set forth in claim 16 wherein the distance from said center point is between 0.3 and 0.5 mm greater than R.

18. The flexible acetabular cup as set forth in claim 15 wherein the cup is made at least in part of polyetheretherketone (PEEK).

19. The flexible acetabular cup as set forth in claim 18 wherein the PEEK is carbon fiber reinforced.

20. The flexible acetabular cup as set forth in claim 18 wherein an inner bearing surface of the cup is made of polyurethane.
